(19) United States
(12) Patent Application Publication
(10) Pub. No.: US 2025/0032784 A1
(43) Pub. Date: Jan. 30, 2025

(54) Medical Electrode Device Comprising at Least One Contact Element

(71) Applicant: BIOTRONIK SE & Co. KG, Berlin (DE)

(72) Inventor: Jens RUMP, Berlin (DE)

(73) Assignee: BIOTRONIK SE & Co. KG, Berlin (DE)

(21) Appl. No.: 18/715,439

(22) PCT Filed: Nov. 24, 2022
(86) PCT No.: PCT/EP2022/083112

§ 371 Date: May 31, 2024

(30) Foreign Application Priority Data
Dec. 14, 2021 (EP) 21214345.7

(51) Int. Cl. A61N 1/05

(52) U.S. Cl. CPC A61N 1/0553; A61N 1/0558

(57) ABSTRACT

A medical electrode device for implantation into a patient comprises a lead body extending along a longitudinal axis and having a proximal end, a distal end and an inner lumen for receiving a mandrel. An electrode end is arranged at the distal end of the lead body and comprises a carrier element and an arrangement of contact elements arranged on the carrier element for contacting tissue. A retaining device is arranged on the electrode end and is configured to interact with the mandrel, wherein the electrode end is adjustable between a folded position and an unfolded position, the retaining device being configured to retain the electrode end in the folded position and being actuatable for causing the electrode end to transfer from the folded position to the unfolded position.

### CROSS-REFERENCE TO RELATED APPLICATIONS

This application is the United States National Phase under 35 U.S.C. § 371 of PCT International Patent Application No. PCT/EP2022/083112, filed on Nov. 24, 2022, which claims the benefit of European Patent Application No. 21214345.7, filed on Dec. 14, 2021, the disclosures of which are hereby incorporated by reference herein in their entireties.

### TECHNICAL FIELD

The present invention relates to a medical electrode device for implantation into a patient according to the preamble of claim 1 and to a method for implanting a medical electrode device into a patient.

### BACKGROUND

A medical electrode device of this kind may, for example, serve for a neuro-stimulation and for this may be implanted into a patient, for example, in the region of the spinal cord, for example into the epidural space near the spinal cord of the spinal column of a patient. In this way a nerve stimulation at the spinal cord may be achieved by injecting electrical stimulation currents.

An electrode device of the kind concerned herein however may also be used for emitting stimulation signals or receiving sense signals at other locations within a patient, for example within the brain or in cardiac applications.

An electrode device of the kind concerned herein comprises a lead body extending along a longitudinal axis and having a proximal end, a distal end and an inner lumen for receiving a mandrel. An electrode end is arranged at the distal end of the lead body and comprises a carrier element and an arrangement of contact elements arranged on the carrier element for contacting tissue. In an implanted state, the lead body with its proximal end is connected to a generator for generating stimulation signals. The electrode end is implanted in a patient, for example in the epidural space of the spinal column, such that the contact elements of the electrode end are in contact with surrounding tissue and may be used to inject stimulation signals into the tissue in order to provide for a stimulation action in the vicinity, e.g., of the spinal cord.

The medical electrode device, for example, has the shape of a so-called paddle electrode, the electrode end having a paddle-like shape carrying, e.g., an arrangement of multiple evenly or unevenly distributed contact elements on its surface for emission of electrical signals into and/or reception of electrical signals from surrounding tissue.

Conventionally, two kinds of electrode devices for a spinal neuro-stimulation exist. Within isodiametric electrodes, contact elements are arranged on a distal portion of the electrode device, the portion of the isodiametric electrode carrying the contact elements having the same diameter as other portions of the electrode device. This allows to percutaneously implant the electrode device using an access to the epidural space in the region of the lumbar spine. For implantation, the electrode device is inserted through vertebral bodies of the spine, and the electrode device is pushed in parallel to the spinal column within the epidural space towards a region of interest at which a therapy shall be performed.

Whereas an electrode device having an isodiametric shape can be pushed through the epidural space, a paddle electrode generally cannot be easily inserted into and pushed through the epidural space, due to the transverse extension of the electrode end of the electrode device carrying the contact elements. Hence, typically a paddle electrode is implanted in a patient by surgically placing the electrode end of the electrode device at the location of interest, for which conventionally portions of vertebral bodies must be removed in order to provide for sufficient space for implanting the electrode end in the epidural space of the spinal column.

There is a general desire to ease an implantation procedure for implanting an electrode device in the shape of a paddle electrode at a location of interest within a patient.

U.S. Pat. No. 6,052,608 discloses an electrode device used in particular for sensing cortical electrical activity. The electrode device herein comprises an arrangement of contact elements having a semi-spherical shape, the contact elements being surrounded by an electrically insulating material of a carrier element.

Different designs of paddle electrodes are known, for example, from U.S. Pat. No. 6,895,283, U.S. Publication No. 2008/0046050 A1, U.S. Publication No. 2014/0172057 A1 and U.S. Pat. No. 9,561,363.

European Publication No. 2 108 398 A1 discloses an implantable medical lead comprising a first paddle portion having a cavity and a second paddle portion having at least one aperture. At least one electrode is electrically connected to at least one conductor. The at least one electrode is placed in the cavity of the first paddle portion and is disposed between the first paddle portion and the second paddle portion. The at least one electrode is disposed in such a way that a portion of the electrode is conductively exposed through the at least one aperture.

The present disclosure is directed toward overcoming one or more of the above-mentioned problems, though not necessarily limited to embodiments that do.

### SUMMARY

It is an object of the instant invention to provide a medical electrode device and a method for implanting a medical electrode device into a patient which allow for an easy procedure for implanting the electrode device in a patient.

At least this object is achieved by means of a medical electrode device comprising the features of claim 1.

Accordingly, the medical electrode device comprises a retaining device arranged on the electrode end and configured to interact with the mandrel, wherein the electrode end is adjustable between a folded position and an unfolded position, the retaining device being configured to retain the electrode end in the folded position and being actuatable for causing the electrode end to transfer from the folded position to the unfolded position.

Typically, when implanting a medical electrode device in the epidural space of the spinal column by pushing the medical electrode device from a point of access upwards through the epidural space, a mandrel is used to guide the electrode device towards a location of interest. The mandrel is received in an inner lumen of the lead body and allows to control movement of the lead body in order to place an end of the lead body with contact elements arranged thereon at a location of interest.

As an electrode end carrying an arrangement of contact elements can potentially not be easily moved through the epidural space of the spinal column, within the instant medical electrode device the electrode end may be adjusted between a folded position and an unfolded position. In the folded position the electrode end may have a radial extension which is smaller than the radial extension in the unfolded position. In particular, in the folded position the electrode end may be deformed such that it is approached towards the longitudinal axis and comprises a radial extension which does not substantially protrude beyond the radial extension of the (isodiametric) lead body. Hence, in the folded position the electrode device may be moved through the epidural space of the spinal column, similarly as for an isodiametric electrode device.

To maintain the electrode end in the folded position, the electrode device comprises a retaining device which is configured to interact with the mandrel. By means of the retaining device the electrode end is held in its folded position while advancing the electrode device, e.g., through the epidural space towards a location of interest, the reduced radial extension of the electrode end allowing to access the epidural space, e.g., in the region of the lumbar spine to then move the electrode device through the epidural space upwards in parallel to the spinal column. Once a location of interest is reached, the retaining device may be actuated using the mandrel such that the electrode end is caused to transfer from the folded position to the unfolded position, the electrode end in the unfolded position having an increased radial extension, e.g., by assuming a generally flat shape in a plane spanned by the longitudinal axis and a direction transverse to the longitudinal axis.

The lead body may, for example, have an isodiametric shape, having a diameter, for example, in a range between 1 mm and 3 mm. In one embodiment, the electrode end in the folded position assumes a diameter which does not exceed the diameter of the lead body.

The inner lumen of the lead body may extend along the entire longitudinal length of the lead body, the mandrel being received within the inner lumen and being movable within the inner lumen along the longitudinal axis. In the folded position of the electrode end the mandrel may, for example, interact with the retaining device for holding the electrode end in the folded position. By withdrawing the mandrel, the retaining device may, for example, be released and in that way actuated such that the electrode end may transfer from the folded position to the unfolded position, for example due to an inherent elasticity of the carrier element of the electrode end, causing the electrode end to assume a generally flat shape in the unfolded position.

In one embodiment, the electrode end comprises a first half on a first side of the longitudinal axis and a second half on a second side of the longitudinal axis opposite the first side. The first half and the second half herein are approached towards one another in the folded position of the electrode end. In the unfolded position, in contrast, the first half and the second half, for example, extend substantially along a common plane such that the electrode end assumes a generally flat, paddle-like shape when it is unfolded at a location of interest. The contact elements herein may be arranged on a surface of the halves such that the contact elements may be brought into abutment with tissue in the surroundings of the electrode end by unfolding the electrode end.

The halves may have a substantially symmetric shape, or may differ from one another in their shape.

The halves may be folded, in the folded position, such that the halves generally extend in parallel to one another, wherein the halves may assume a straight or a curved shape (when viewed in a cross-section perpendicular to the longitudinal axis). In another embodiment, the halves each may be folded once or multiple times, such that the electrode end, in the folded position, assumes a zigzag shape with each half being folded once or multiple times.

In one embodiment, the retaining device is formed by at least one fixation element arranged on the carrier element and configured to interact with the mandrel in the folded position of the electrode end. The fixation element may, for example, form a hook or a loop shaped to interact with the mandrel, wherein the mandrel in the folded position of the electrode end engages with the at least one fixation element of the electrode end such that the electrode end is retained in its folded state.

The fixation element herein may be rigid or may be flexibly deformable. A rigid fixation element may, for example, have the shape of a sleeve made from a plastics or metal material. A flexibly deformable fixation element may, for example, be formed by a flexibly deformable tab made from a plastics material or from a metal material. A plastics tab may, for example, be integrally formed with the material of the carrier element. A metal tab may, for example, be formed by a metal wire arranged on the carrier element.

In an embodiment in which the electrode end comprises two halves arranged on different sides of the longitudinal axis, each half may comprise an arrangement of fixation elements configured to interact with the mandrel for retaining the halves in the folded position of the electrode end. Herein, the fixation elements of the halves may alternate along the longitudinal axis in that a fixation element of one half is followed, when viewed along the longitudinal axis, by a fixation element of the other half. The fixation elements of the two halves hence are arranged in an alternating fashion along the longitudinal axis, wherein in the folded position the fixation elements are aligned along the longitudinal axis to interact with the mandrel extending through the fixation elements. By removing the mandrel, then, the fixation elements may be released such that the electrode end may transfer from the folded position to the unfolded position during the implantation.

In one embodiment, each half comprises an arrangement of recesses for receiving the fixation elements of the respective other half therein in the folded position of the electrode end. By providing recesses on the halves in which the fixation elements of the respective other half come to rest in the folded position of the electrode end, the radial extension of the electrode end in the folded position may be further reduced, in that the fixation elements may be space-efficiently received in between the halves of the electrode end for interaction with the mandrel. Each fixation element on one half herein may be associated with a recess on the respective other half, wherein in the folded position the fixation element is partially received within the associated recess.

In one embodiment, the retaining device is formed by a wire configured to interact with the mandrel for exerting a pulling force on the carrier element in the folded position of the electrode end. In the folded position the mandrel may, for example, tension the pull wire such that, by means of the pull wire, halves of the electrode end are pulled towards one another in order to retain the electrode end in the folded position. By releasing the pull wire, the pulling force may be reduced or canceled such that the halves of the electrode end may transition towards the unfolded position due to the inherent elasticity of the carrier element.

The pull wire may, for example, comprise sections which transversely extend in between edges of the electrode end spaced apart along a transverse direction with respect to the longitudinal axis. By means of the pull wire a pulling force in a generally transverse direction with respect to the longitudinal axis may be introduced into the electrode end, such that the halves of the electrode end are pulled towards one another in the folded position of the electrode end.

Other sections of the pull wire may extend along the axis of the electrode end, such that a closed-loop pull wire is formed, the pull wire being configured to interact with the mandrel in order to retain the electrode end in the folded position and to cause the electrode end to unfold once a location of interest is reached.

In one embodiment, the carrier element comprises a protruding web extending along the longitudinal axis and forming an opening for guiding the mandrel therein. Whereas halves of the electrode end carrying the contact elements may generally extend, in the unfolded position of the electrode end, along a common plane spanned by the longitudinal axis and a transverse direction with respect to the longitudinal axis, the web may protrude from the halves along a height direction perpendicular to the plane of the halves of the electrode end. The web forms an opening for guiding the mandrel, the web having an arrangement of gap openings in which fixation elements, e.g., in the shape of loops or hooks of the halves may be placed for interacting with the mandrel guided within the web. In the folded position, hence, the halves of the electrode end are approached towards the web of the electrode end, wherein by withdrawing the mandrel the retaining device may be released in order to cause the electrode end to transition towards its unfolded position.

In one embodiment, a system for implantation into a patient comprises a medical electrode device of the kind described above and a mandrel received in the inner lumen of the lead body. The mandrel in particular may be movable with respect to the lead body of the medical electrode device to actuate the retaining device for causing the electrode end to transfer from the folded position to the unfolded position. The retaining device may in particular be actuated by withdrawing the mandrel, such that the retaining device is released and the electrode end may transfer from the folded position to the unfolded position due to the inherent elasticity of the carrier element.

In another aspect, a method for implanting a medical electrode device into a patient comprises: providing a medical electrode device comprising a lead body extending along a longitudinal axis and having a proximal end, a distal end and an inner lumen receiving a mandrel therein, and an electrode end arranged at the distal end of the lead body and comprising a carrier element and an arrangement of contact elements arranged on the carrier element for contacting tissue. The method further comprises: retaining the electrode end in a folded position using a retaining device arranged on the electrode end and configured to interact with the mandrel; and causing the electrode end to transfer from the folded position to an unfolded position by actuating the retaining device using the mandrel received in the inner lumen of the lead body.

The advantages and advantageous embodiments described above for the medical electrode device equally apply also to the method such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily understood with reference to the following detailed description and the embodiments shown in the drawings. Herein,

FIG. 1 shows a view of an electrode device connected to a stimulation device in an implanted state in the area of the spine of a patient;

FIG. 2 shows a view of the electrode device in the epidural space in the region of the spinal column;

FIG. 3 shows a view of an electrode end of an embodiment of an electrode device;

FIG. 4A shows a schematic view of an electrode end of a medical electrode device, in an unfolded position;

FIG. 4B shows the electrode end interacting with a mandrel for retaining the electrode end in a folded position;

FIG. 5A shows a view of an embodiment of an electrode device, with an electrode end in a folded position;

FIG. 5B shows the electrode end during unfolding;

FIG. 5C shows the electrode end in an unfolded position;

FIG. 5D shows an electrode arrangement on the electrode end in the unfolded position;

FIG. 5E shows a cross-sectional view of the arrangement of FIG. 5A;

FIG. 5F shows a cross-sectional view of the arrangement of FIG. 5C;

FIG. 6A shows a view of another embodiment of an electrode device, with an electrode end in a folded position;

FIG. 6B shows the electrode end during unfolding;

FIG. 6C shows the electrode end in an unfolded position;

FIG. 6D shows an electrode arrangement on the electrode end in the unfolded position;

FIG. 6E shows a cross-sectional view of the arrangement of FIG. 6A;

FIG. 6F shows a cross-sectional view of the arrangement of FIG. 6C;

FIG. 7A shows a view of yet another embodiment of an electrode device, with an electrode end in a folded position;

FIG. 7B shows the electrode end during unfolding;

FIG. 7C shows the electrode end in an unfolded position;

FIG. 7D shows an electrode arrangement on the electrode end in the unfolded position;

FIG. 7E shows a cross-sectional view of the arrangement of FIG. 7A;

FIG. 7F shows a cross-sectional view of the arrangement of FIG. 7C;

FIG. 8A shows a view of yet another embodiment of an electrode device, with an electrode end in a folded position;

FIG. 8B shows the electrode end during unfolding;

FIG. 8C shows the electrode end in an unfolded position;

FIG. 8D shows an electrode arrangement on the electrode end in the unfolded position;

FIG. 8E shows a cross-sectional view of the arrangement of FIG. 8A;

FIG. 8F shows a cross-sectional view of the arrangement of FIG. 8C;

FIG. 9A shows a view of yet another embodiment of an electrode device, with an electrode end in a folded position;

FIG. 9B shows the electrode end during unfolding;

FIG. 9C shows the electrode end in an unfolded position;

FIG. 9D shows an electrode arrangement on the electrode end in the unfolded position;

FIG. 9E shows a cross-sectional view of the arrangement of FIG. 9A;

FIG. 9F shows a cross-sectional view of the arrangement of FIG. 9B;

FIG. 9G shows a cross-sectional view of the arrangement of FIG. 9D;

FIG. 10A shows a view of yet another embodiment of an electrode device, with an electrode end in a folded position:

FIG. 10B shows the electrode end during unfolding:

FIG. 10C shows the electrode end in an unfolded position:

FIG. 10D shows an electrode arrangement on the electrode end in the unfolded position:

FIG. 10E shows a cross-sectional view of the arrangement of FIG. 10A; and

FIG. 10F shows a cross-sectional view of the arrangement of FIG. 10C.

### DETAILED DESCRIPTION

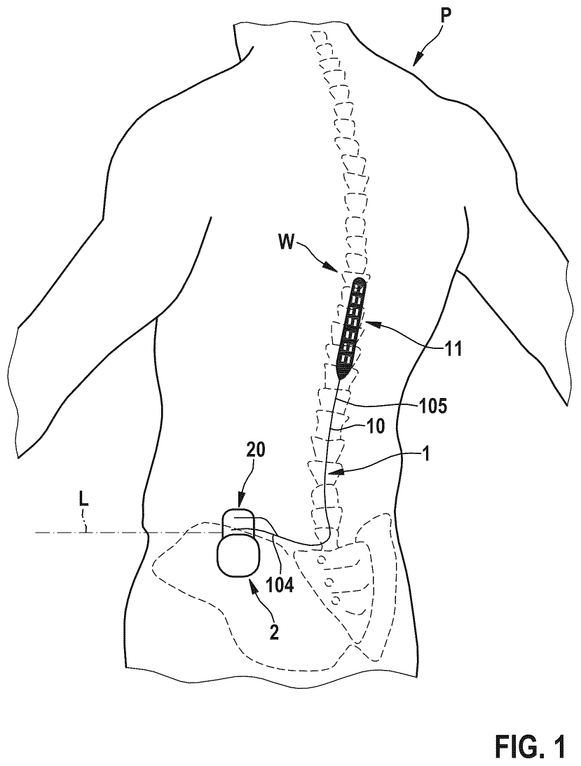

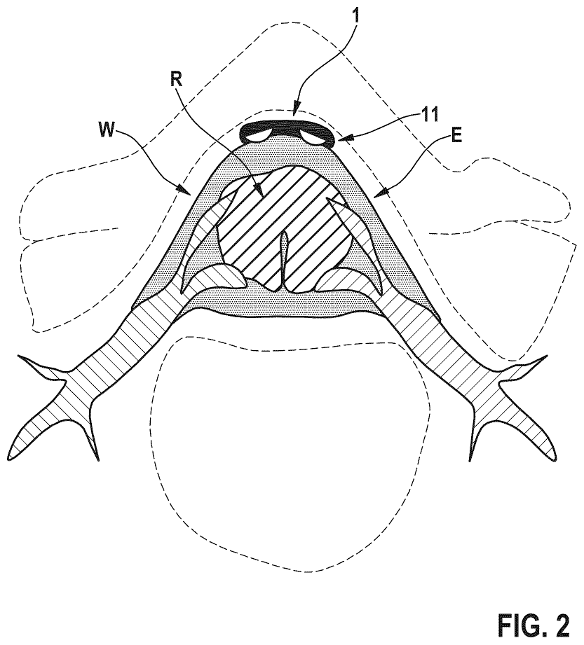

An electrode device 1, as shown in an embodiment in FIGS. 1 and 2, is formed as a so-called paddle electrode and comprises a lead body 10 and an electrode end 11 connected to the lead body 10 at a distal end 105 of the lead body 10, a plurality of contact elements being attached to the electrode end 11 for injecting an electrical current, e.g., in the region of the spinal column W of a patient P.

The electrode device 1 at a proximal end 104 of the lead body 10 is connected to a connector block 20 of a stimulation device 2, via which stimulation currents can be delivered to the electrode device 1 and output via the electrode arrangement arranged on the electrode end 11 to stimulate the spinal cord R in the region of the spinal column W.

As can be seen from the sectional view of FIG. 2, in the embodiment shown the electrode device 1 is implanted with the electrode end 11 in the epidural space E in the region of the spinal column W of the patient P in such a way that the electrode end 11 is located in the region of the spinal cord R and can thus introduce stimulation currents in a directed manner into the spinal cord R in order to effect nerve stimulation in the region of the spinal cord R.

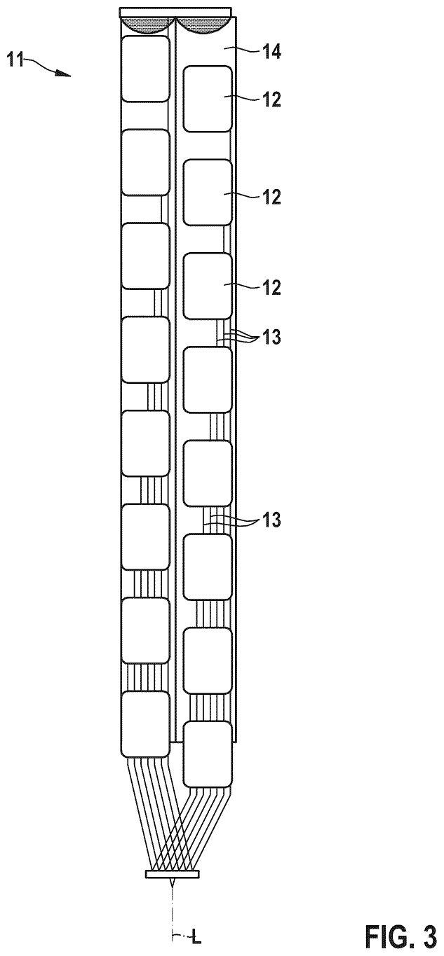

While the lead body 10, for example, comprises a circular (isodiametric) cross-section, the electrode device 1 is flattened in the area of the electrode end 11 which, as can be seen in FIG. 3, carries a plurality of contact elements 12 evenly or unevenly spaced on the electrode end 11 in such a way that stimulation energy can be fed in a directed manner, for example, into the spinal cord R of a patient P.

As further illustrated in FIG. 3, each contact element 12 is connected to a supply line 13, wherein each contact element 12, for example, is connected to the stimulation device 2 via an associated, individual supply line 13 and thus may be supplied with stimulation energy via the stimulation device 2 to emit electrical signals. The supply lines 13 are jointly routed as a cable strand in the lead body 10 in an encapsulated manner to the stimulation device 2.

The contact elements 12 are arranged on a carrier element 14 and are exposed with a surface facing outwards and can therefore come into contact with surrounding tissue when the electrode device 1 is implanted in a patient.

A medical electrode device 1 having a flattened electrode end 11 arranged on a lead body 10, as is shown in FIGS. 1 to 3, conventionally is implanted in a patient P in a surgical procedure in which the electrode end 11 surgically is placed at a location of interest. A surgical procedure herein in particular is necessary if the electrode end 11 with its flattened shape cannot be moved upwards through the epidural space E from an access point in the region of the lumbar spine.

In order to ease implantation of an electrode device 1 having a flattened electrode end 11, it herein is proposed to form the electrode end 11 such that it is foldable between a folded position and an unfolded position. In the folded position the radial extension of the electrode end 11 may be reduced, in particular in that the electrode end 11 does not protrude radially beyond the isodiametric lead body 10. The electrode end 11 may be implanted in its folded position and may be unfolded at a location of interest in order to bring contact elements 12 arranged on a carrier element 14 of the electrode end 11 into electrical contact with surrounding tissue in order to be able to inject stimulation currents at the location of interest.

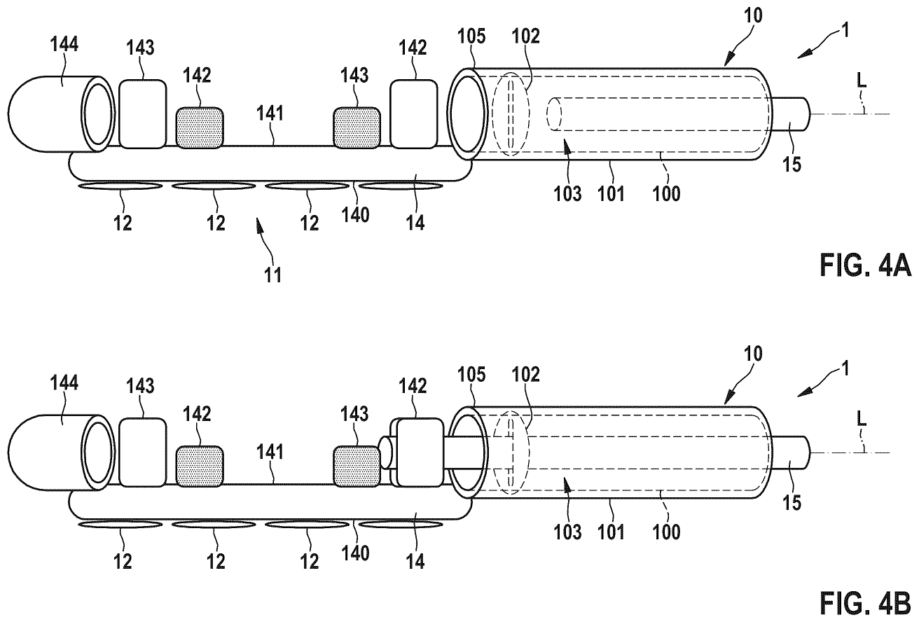

Referring now to FIGS. 4A and 4B, the electrode device 1 comprises a lead body 10 having an outer tube 101 and an inner tube 100 forming a space therebetween. Supply lines 13 in the shape of electrical wires (e.g., DFT wires) are arranged within the space in between the inner tube 100 and the outer tube 101 in order to electrically supply contact elements 12 arranged on a carrier element 14 of an electrode end 11 arranged on the lead body 10.

The outer tube 101 may, for example, have an outer diameter of 2 mm and an inner diameter of 1.5 mm. The inner tube 100 may have an outer diameter of 0.7 mm and an inner diameter of 0.45 mm.

The inner tube 100 forms an inner lumen 103 extending longitudinally within the inner tube 100. The lead body 10 extends longitudinally along a longitudinal axis L, wherein the lead body 10 is flexible such that the longitudinal axis L may be curved along the extension of the lead body 10. The inner lumen 103 extends longitudinally along the longitudinal axis L within the inner tube 100 along the entire length of the lead body 10.

For implantation, a mandrel 15 is received within the inner tube 100, the mandrel 15 extending through the lead body 10 from the proximal end 104 to the distal end 105, the mandrel 15 in an implantation state exiting from the lead body 10 at the distal end 105 towards the electrode end 11.

The inner tube 100 is open at the distal end 105 of the lead body 10. In order to prevent fluid to enter into the lead body 10 at the distal end 105, a sealing element 102 is arranged within the inner tube 100 at the distal end 105, the mandrel 15 extending through the sealing element 102, as it is visible from FIG. 4B.

The electrode end 11 in an operative state assumes an unfolded position in which the electrode end 11 comprises a generally flat shape, the contact elements 12 being arranged on a face 140 which, in the arrangement of FIGS. 4A, 4B, points outwards such that the contact elements 12 may be brought into electrical contact with surrounding tissue.

At a face 141 opposite the face 140 a retaining device in the shape of fixation elements 142, 143 is arranged on the carrier element 14, the fixation elements 142, 143 being configured to interact, during implantation of the electrode device 1, with the mandrel 15 in order to retain the electrode end 11 in a folded position in which the radial extension of the electrode end 11 is reduced in comparison to the unfolded position.

As it shall be explained in further detail below, the fixation elements 142, 143 are arranged on halves of the electrode end 11. An end element 144 in the shape of a cup-like sleeve is placed at a distal end of the electrode end 11 for receiving the mandrel 15 therein in the folded position of the electrode end 11 during implantation of the electrode device 1.

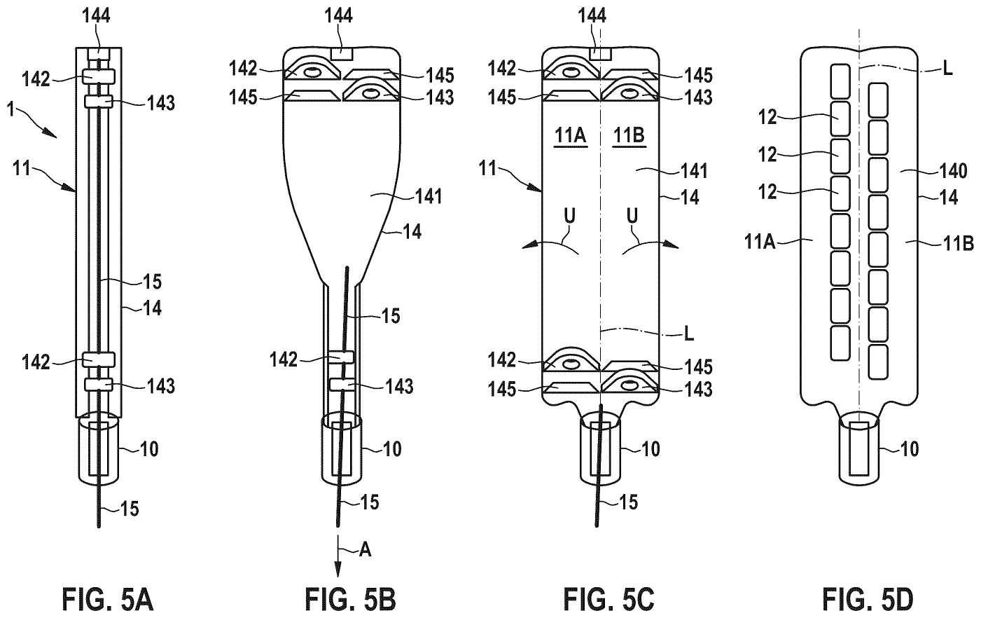

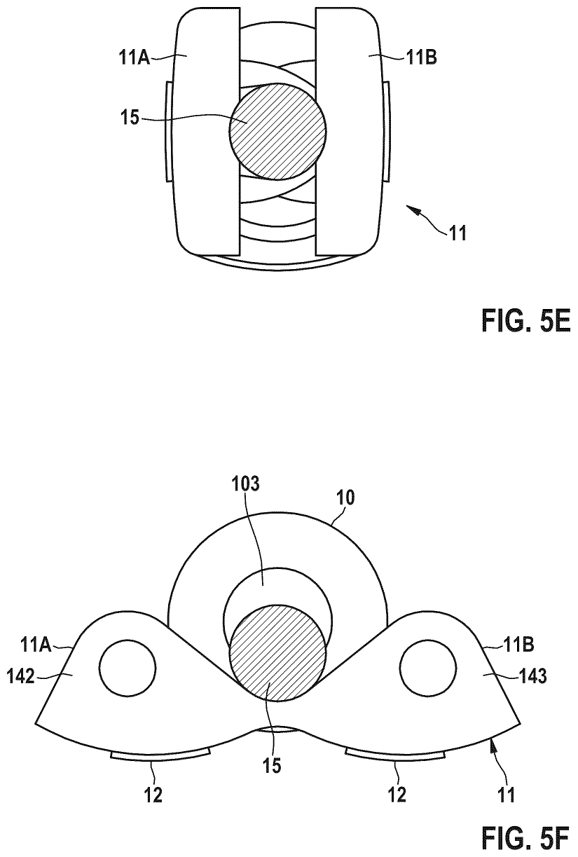

Referring now to FIGS. 5A to 5F, in one embodiment the electrode end 11 comprises halves 11A, 11B, the halves 11A, 11B being logically separated from one another by the longitudinal axis L along which the lead body 10 extends. Each half 11A, 11B carries an arrangement of contact elements 12, as visible from FIG. 5D, and an arrangement of fixation elements 142, 143, as visible from FIG. 5C, the contact elements 12 and the fixation elements 142, 143 being placed on opposite faces 140, 141 of the carrier element 14 of the electrode end 11.

In a folded position, as illustrated in FIGS. 5A and 5E, the halves 11A, 11B of the electrode end 11 are folded in that the halves 11A, 11B are approached towards one another. The radial extension of the electrode end 11 hence is reduced, such that the electrode end 11 does not substantially protrude from the isodiametric lead body 10.

In the folded position the mandrel 15 is introduced through the lead body 10 to extend across the electrode end 11 such that a distal end of the mandrel 15 is received in the end element 144, as visible from FIG. 5A. The mandrel 15 herein extends through the fixation elements 142, 143, which have the shape of loops, such that the halves 11A, 11B by means of the fixation elements 142, 143 are held on the mandrel 15 and hence are retained in the folded position.

In the folded position the electrode device 1 may be advanced through the epidural space E upwards in parallel to the spinal column from an access point in the region of the lumbar spine, as visible from FIG. 1, for advancing the electrode end 11 towards a location of interest. Once the location of interest is reached, the electrode end 11 may be unfolded by withdrawing the mandrel 15 in a withdrawal direction A, as it is illustrated in FIG. 5B. The mandrel 15 hence is disengaged from the fixation elements 142, 143, such that the halves 11A, 11B of the electrode end 11 may unfold in an unfolding direction U due to the inherent elasticity of the carrier element 14 of the electrode end 11.

By withdrawing the mandrel 15, the electrode end 11 hence is caused to unfold, such that the electrode end 11 assumes a generally flat shape and the contact elements 12 at the face 140 of the carrier element 14 are brought into contact with tissue at the location of interest.

In the embodiment of FIGS. 5A to 5F the fixation elements 142, 143 may be integrally formed with the material of the carrier element 14, or may be formed as separate elements, for example, from a metal material (such as platinum or MP35N) on the carrier element 14. The fixation elements 142, 143 may be flexibly deformable, or may be rigid.

The fixation elements 142, 143 herein are displaced with respect to one another along the longitudinal axis L, such that the fixation elements 142, 143 may be brought into engagement with the mandrel 15 for implanting the electrode device 1.

The fixation elements 142, 143 may, for example, form a loop opening having a diameter of 0.45 mm, the openings being elevated from the face 141 of the carrier element 14, e.g., by 0.55 mm.

As visible from FIG. 5C, each fixation element 142, 143 of each half 11A, 11B is associated with a corresponding recess 145 on the respective other half 11B, 11A, the recesses 145 being formed such that they partially receive the fixation elements 142, 143 therein in the folded state of the electrode end 11, hence allowing to further reduce the radial extension in the folded position of the electrode end 11.

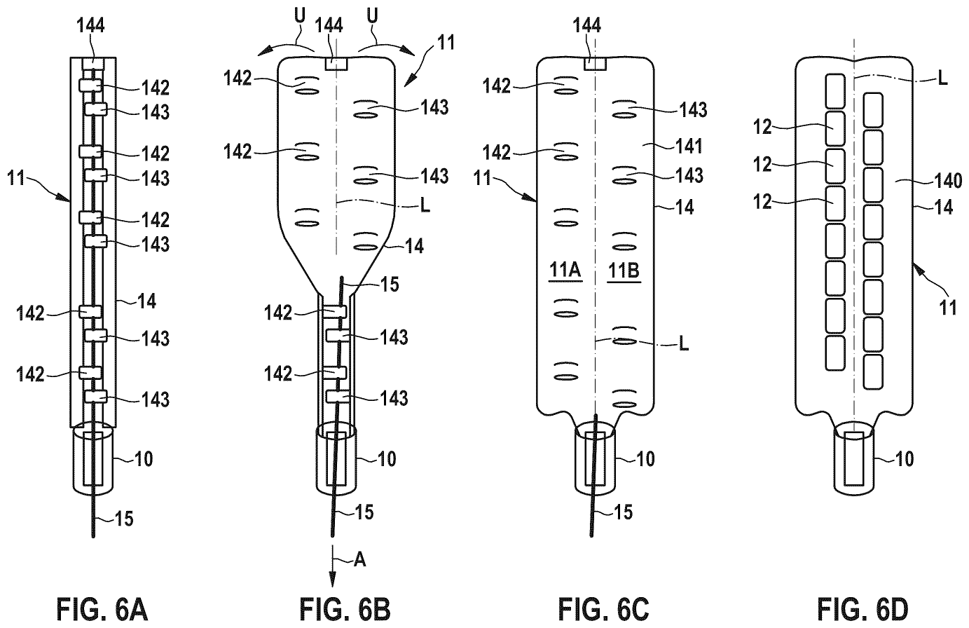

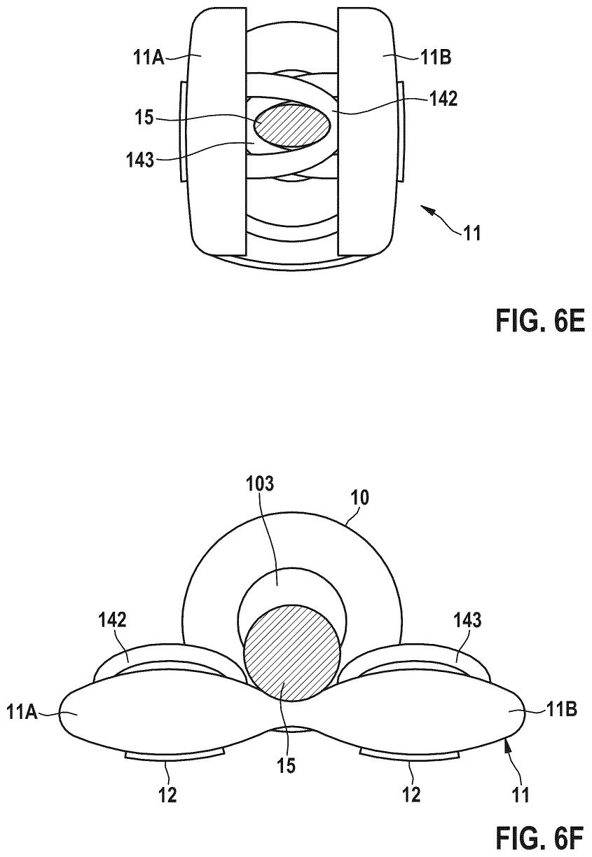

Referring now to FIGS. 6A to 6F, in another embodiment the fixation elements 142, 143 may be formed as flexibly deformable tabs, wherein a multiplicity of fixation elements 142, 143 may be arranged on each half 11A, 11B of the electrode end 11. The fixation elements 142, 143 on the two halves 11A, 11B are arranged to alternate along the longitudinal axis L, a fixation element 142 on one half 11A being followed, when viewed along the longitudinal axis L, by a fixation element 143 on the other half 11B and vice versa.

Other than that, the formation of the fixation elements 142, 143 in the embodiment of FIGS. 6A to 6F is functionally identical to the embodiment of FIGS. 5A to 5F, such that it also shall be referred to the above. Again, by withdrawing the mandrel 15 the fixation elements 142, 143 may be released such that the electrode end 11 with its halves 11A, 11B is able to unfold and assumes a generally flat position in which the contact elements 12 arranged on the face 140 of the carrier element 14 of the electrode end 11 may be in electrical contact with surrounding tissue.

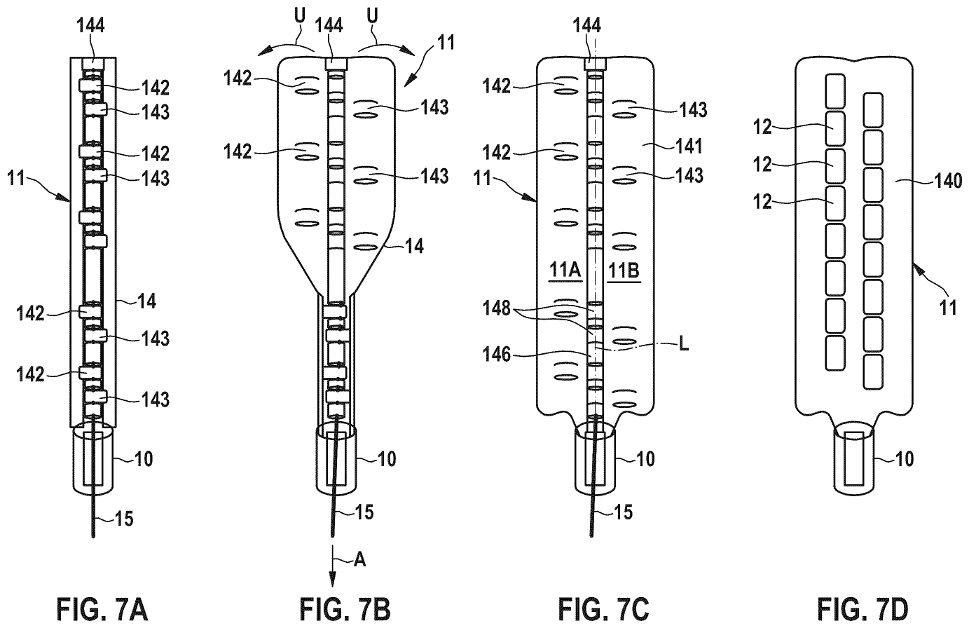

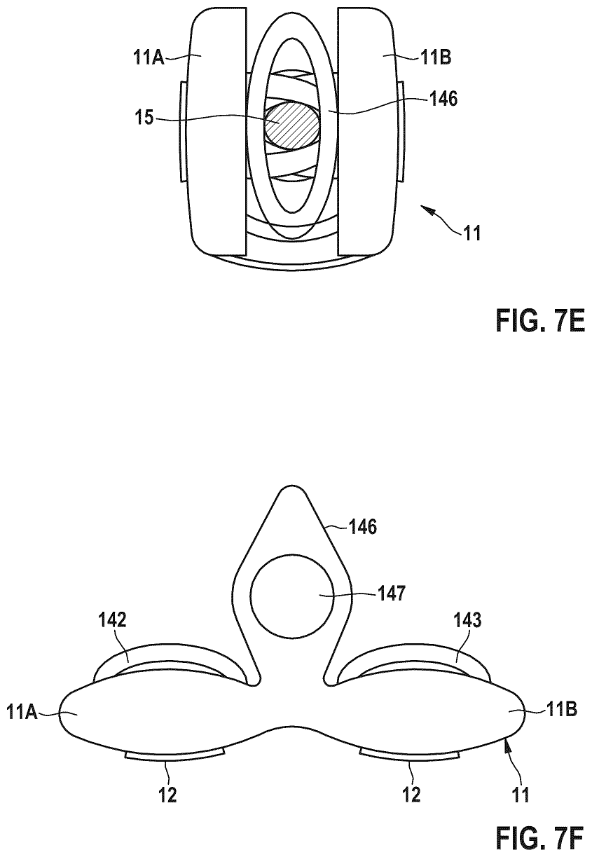

Referring now to FIGS. 7A to 7F, in another embodiment a protruding web 146 is formed on the carrier element 14, the protruding web 146 extending axially along the longitudinal axis L and protruding from the carrier element 14 in a direction perpendicular to the generally flat plane of extension of the halves 11A, 11B of the carrier element 14. The protruding web 146 is integrally formed from the material of the carrier element 14, the protruding web 146 having a central opening 147 through which the mandrel 15 is guided in the folded position of the electrode end 11 (see FIGS. 7A and 7F).

The protruding web 146 forms gap openings 148 into which the fixation elements 142, 143 of the halves 11A, 11B of the electrode end 11 reach in the folded position, such that the fixation elements 142, 143 are aligned along the longitudinal axis L and the mandrel 15, in the folded position, extends through the fixation elements 142, 143, as shown in FIG. 7A. By withdrawing the mandrel 15, as shown in FIG. 7B, the mandrel 15 is brought out of engagement from the fixation elements 142, 143, such that the halves 11A, 11B are caused to unfold in an unfolding direction U and the electrode end 11 assumes its generally flat, unfolded position, as shown in FIGS. 7C, 7D and 7F.

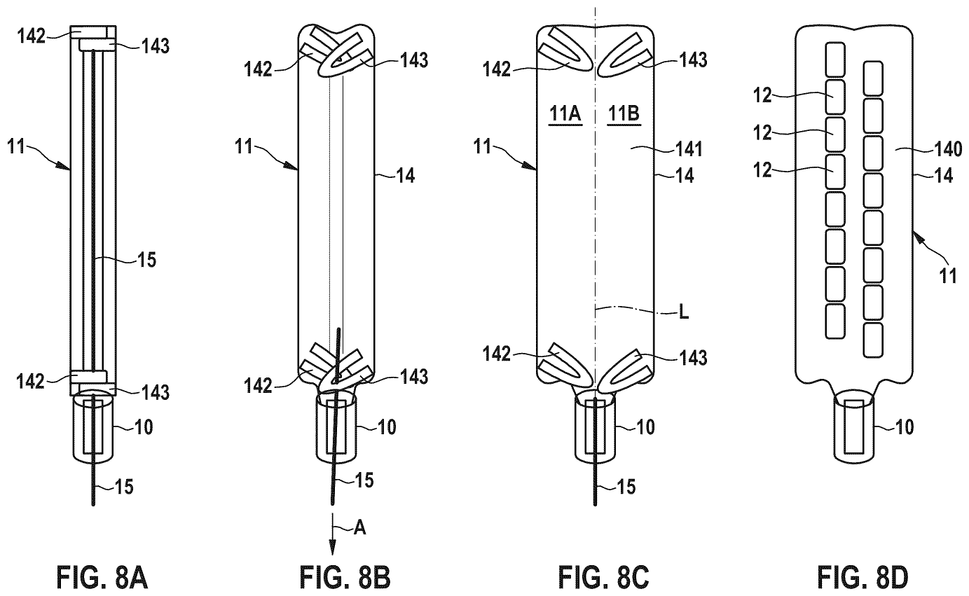

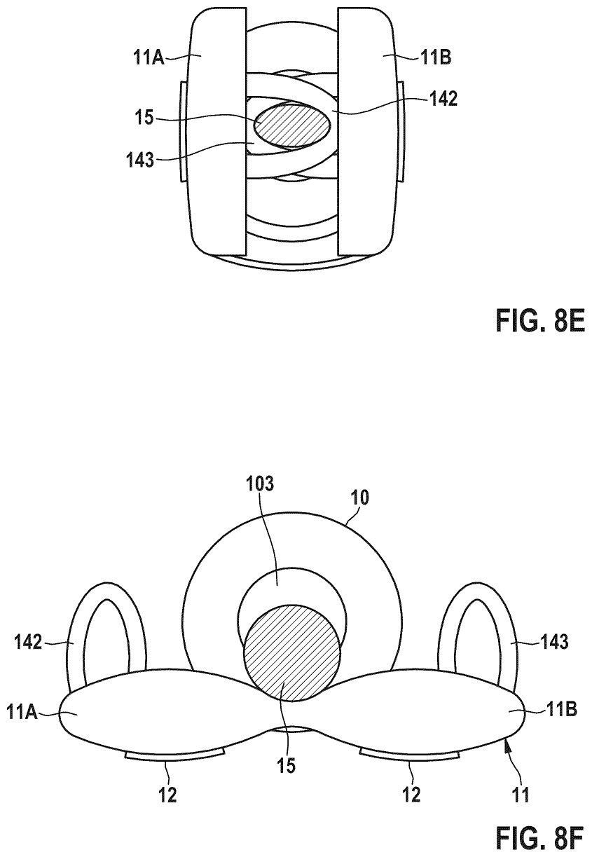

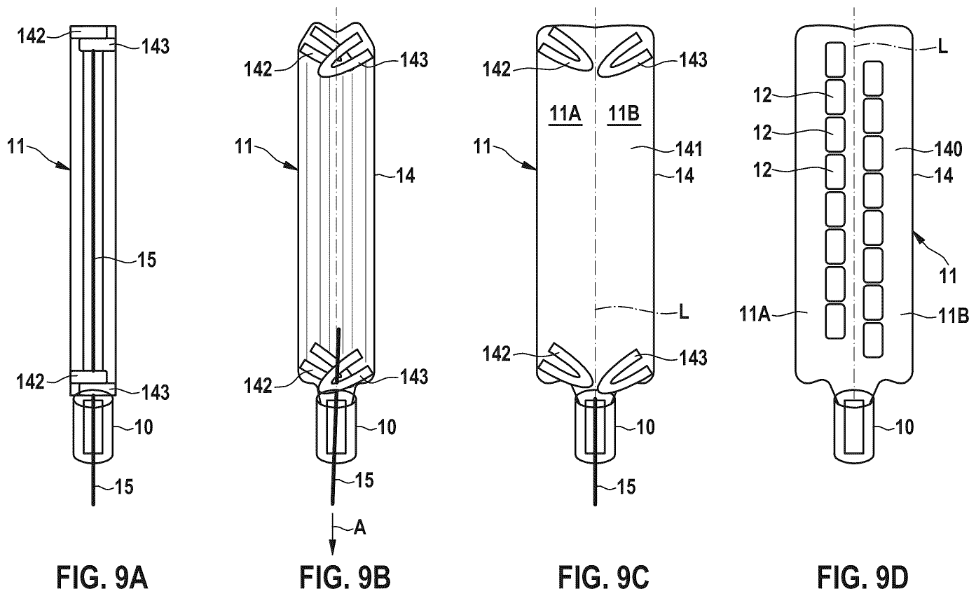

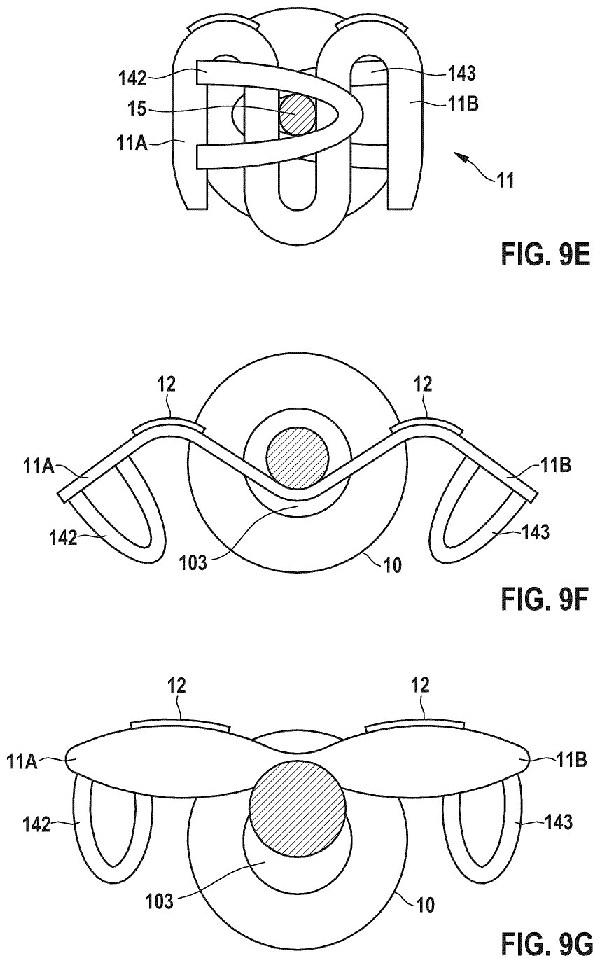

In yet another embodiment, shown in FIGS. 8A to 8F and 9A to 9G, fixation elements 142, 143 are formed as protruding, flexible tabs at the two axial ends of the electrode end 11.

Herein—as shown in FIGS. 8A to 8F—the halves 11A, 11B of the electrode end 11 may be approached towards one another to substantially extend in parallel to one another in the folded position, or—as shown in FIGS. 9A to 9G—the halves 11A, 11B may each be folded in themselves such that the electrode end 11, in the folded position, assumes a zigzag shape in a cross-sectional view perpendicular to the longitudinal axis L, as it is visible from FIGS. 9E and 9F.

Generally, the electrode end 11 may assume an arbitrary folding pattern, the halves 11A, 11B not being folded in themselves or being folded in themselves about one or multiple folding lines once or multiple times in the folded position of the electrode end 11.

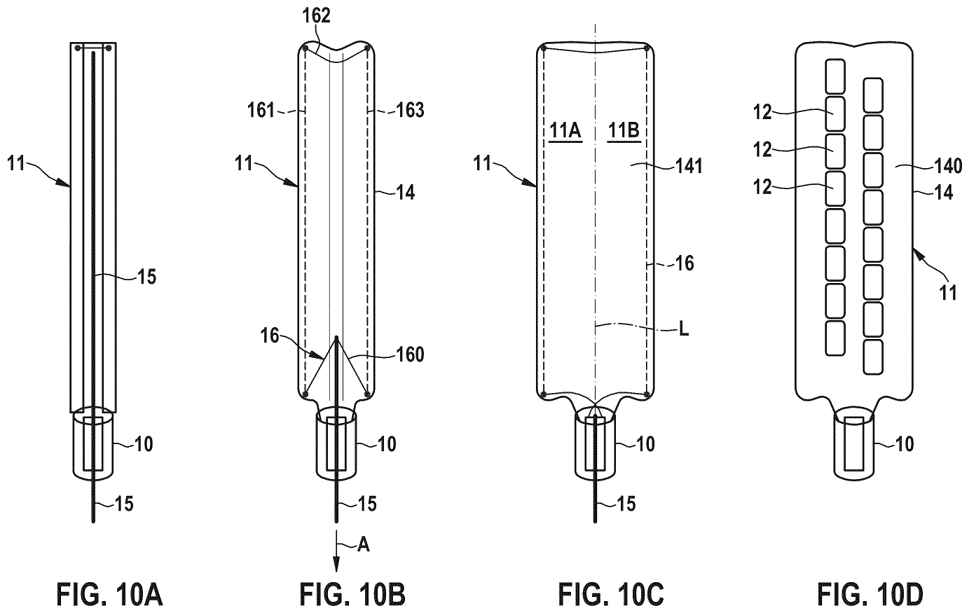

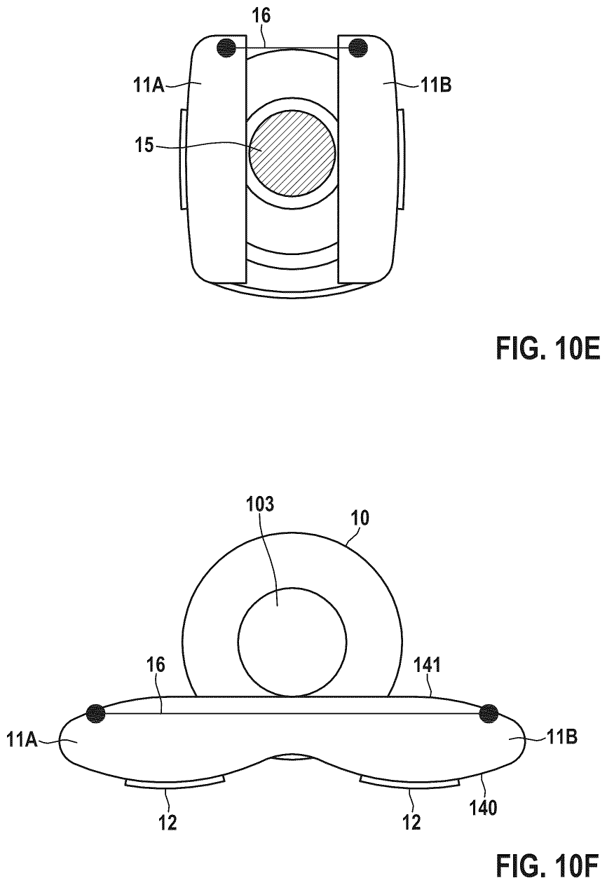

Referring now to FIGS. 10A to 10F, in another embodiment a retaining device 16 may have the shape of a pull wire having sections 160, 161, 162, 163 forming a closed loop on the carrier element 14. Sections 161, 163 herein extend along longitudinal, axial edges of the carrier element 14. Sections 160, 162 in turn extend transversely across the carrier element 14 at axially opposite ends of the carrier element 14.

Herein, the sections 160, 162 may extend on the face 141 of the carrier element 14, whereas the sections 161, 163 extend on the opposite face 140, the pull wire extending through openings in the carrier element 14 at transitional points between the sections 160-163.

In the embodiment of FIGS. 10A to 10F, the mandrel 15 is used to tension the pull wire 16 such that, in the folded position, the halves 11A, 11B are pulled towards one another by means of a pulling force exerted through the pull wire 16, as visible from FIGS. 10A and 10E. By withdrawing the mandrel 15 in the withdrawing direction A the tensioning force on the pull wire 16 is released, such that the halves 11A, 11B are caused to unfold, as visible from the transition between FIGS. 10B and 10C.

The idea underlying the instant invention is not limited to the embodiments described above, but may be implemented in an entirely different fashion.

The electrode end may comprise an arrangement of contact elements with any number of contact elements, for example in between 4 to 16 contact elements.

Each contact element may be associated with an individual supply line, the supply lines being arranged within the lead body to electrically connect the contact elements on the distal electrode end to a generator device at the proximal end of the lead body.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable electrode device
10 Lead body
100 Inner tube
101 Outer tube
102 Sealing element
103 Inner lumen
104 Proximal end
105 Distal end
11 Electrode end
11A, 11B Halves
12 Contact element
13 Supply line
14 Carrier element
140, 141 Face
142, 143 Retaining device (fixation element)
144 End element
145 Recess
146 Protruding web
147 Opening
148 Gap opening
15 Mandrel
16 Retaining device (pull wire)
160-163 Sections
2 Stimulation device
20 Connector block
A Withdrawal direction
E Epidural space
L Longitudinal axis
P Patient
R Spinal cord
U Unfolding direction
W Spinal column

1. A medical electrode device for implantation into a patient, comprising:
a lead body extending along a longitudinal axis and having a proximal end, a distal end and an inner lumen for receiving a mandrel; and
an electrode end arranged at the distal end of the lead body and comprising a carrier element and an arrangement of contact elements arranged on the carrier element for contacting tissue;
wherein a retaining device arranged on the electrode end and configured to interact with the mandrel, wherein the electrode end is adjustable between a folded position and an unfolded position, the retaining device being configured to retain the electrode end in the folded position and being actuatable for causing the electrode end to transfer from the folded position to the unfolded position.

2. The medical electrode device according to claim 1, wherein the electrode end, in the folded position, comprises a first radial extension with respect to the longitudinal axis and, in the unfolded position, comprises a second radial extension with respect to the longitudinal axis larger than the first radial extension.

3. The medical electrode device according to claim 1, wherein the electrode end comprises a first half on a first side of the longitudinal axis and a second half on a second side of the longitudinal axis opposite the first side, wherein the first half and the second half are approached towards one another in the folded position of the electrode end and substantially extend along a common plane in the unfolded position of the electrode end.

4. The medical electrode device according to claim 1, wherein the retaining device is formed by at least one fixation element arranged on the carrier element and configured to interact with the mandrel in the folded position of the electrode end.

5. The medical electrode device according to claim 4, wherein the fixation element forms a loop for interacting with the mandrel.

6. The medical electrode device according to claim 4, wherein the fixation element is formed as a rigid element or is flexibly deformable.

7. The medical electrode device according to claim 4, wherein halves of the electrode end on different sides of the longitudinal axis each comprise an arrangement of fixation elements configured to interact with the mandrel for retaining the halves in the folded position of the electrode end.

8. The medical electrode device according to claim 7, wherein the fixation elements of the halves alternate along the longitudinal axis in that a fixation element of one half is followed, when viewed along the longitudinal axis, by a fixation element of the other half.

9. The medical electrode device according to claim 7, wherein each half comprises an arrangement of recesses for receiving the fixation elements of the respective other half therein in the folded position of the electrode end.

10. The medical electrode device according to claim 1, wherein the retaining device is formed by a pull wire configured to interact with the mandrel for exerting a pulling force on the carrier element in the folded position of the electrode end.

11. The medical electrode device according to claim 10, wherein the pull wire comprises at least one section extending in between edges of the electrode end spaced apart along a transverse direction with respect to the longitudinal axis to introduce a pulling force transverse to the longitudinal axis in the folded position of the electrode end.

12. The medical electrode device according to claim 1, wherein the carrier element comprises a protruding web extending along the longitudinal axis and forming an opening for guiding the mandrel, the protruding web having at least one gap opening in which the retaining device is received in the folded position of the electrode end for interacting with the mandrel.

13. A system for implantation into a patient, comprising: a medical electrode device according to claim 1; and a mandrel received in the inner lumen of the lead body.

14. The system according to claim 13, wherein the mandrel is movable with respect to the lead body of the medical electrode device to actuate the retaining device for causing the electrode end to transfer from the folded position to the unfolded position.

15. A method for implanting a medical electrode device into a patient, comprising:
providing a medical electrode device comprising
a lead body extending along a longitudinal axis and having a proximal end, a distal end and an inner lumen receiving a mandrel therein, and
an electrode end arranged at the distal end of the lead body and comprising a carrier element and an arrangement of contact elements arranged on the carrier element for contacting tissue;
wherein retaining the electrode end in a folded position using a retaining device arranged on the electrode end and configured to interact with the mandrel; and
causing the electrode end to transfer from the folded position to an unfolded position by actuating the retaining device using the mandrel received in the inner lumen of the lead body.
